(19) United States
(12) Patent Application Publication
(10) Pub. No.: US 2020/0029817 A1
(43) Pub. Date: Jan. 30, 2020

(54) CARDIAC MAPPING SYSTEMS, METHODS, AND KITS INCLUDING FIDUCIAL MARKERS

(71) Applicant: CATHETER PRECISION, INC., Ledgewood, NJ (US)

(72) Inventor: Steve ADLER, Randolph, NJ (US)

(21) Appl. No.: 16/523,353

(22) Filed: Jul. 26, 2019

Related U.S. Application Data
(60) Provisional application No. 62/711,777, filed on Jul. 30, 2018.

(51) Int. Cl. A61B 5/00; A61B 90/00; A61B 5/055; A61B 6/00; A61B 6/03; G06T 17/00

(52) U.S. Cl. CPC A61B 5/0044; A61B 5/0077; A61B 90/39; A61B 5/0035; G06T 2210/41; A61B 6/5247; A61B 6/032; G06T 17/00; A61B 2090/3937; A61B 5/055

(57) ABSTRACT

Various embodiments provide a cardiac imaging system, method, and kit, the system including fiducial markers configured to be placed on a patient and configured to be recognizable using image processing, an external imaging device configured to generate image data of a patient's body including the fiducial markers and electrocardiogram (ECG) leads, and a processing unit configured to identify anatomical locations corresponding to the fiducial markers by detecting light reflected from the fiducial markers included in the image data and use the identified locations to merge the image data with a 3D anatomical model of the patient's chest.

### CROSS-REFERENCE TO RELATED APPLICATION

This application claims the benefit of priority to U.S. Provisional Patent Application No. 62/711,777, entitled “CARDIAC MAPPING SYSTEMS, METHODS, AND KITS INCLUDING FIDUCIAL MARKERS”, filed Jul. 30, 2018, the entire contents of both of which are hereby incorporated by reference for all purposes.

### BACKGROUND

Some heart defects in the conduction system result in asynchronous contraction (arrhythmia) of the heart and are sometimes referred to as conduction disorders. As a result, the heart does not pump enough blood, which may ultimately lead to heart failure. Conduction disorders can have a variety of causes, including age, heart (muscle) damage, medications and genetics.

Premature Ventricular Contractions (PVCs) are abnormal or aberrant heart beats that start somewhere in the heart ventricles rather than in the upper chambers of the heart as with normal sinus beats. PVCs typically result in a lower cardiac output as the ventricles contract before they have had a chance to completely fill with blood. PVCs may also trigger Ventricular Tachycardia (VT or V-Tach).

Ventricular tachycardia (VT or V-Tach) is another heart arrhythmia disorder caused by abnormal electrical signals in the heart ventricles. In VT, the abnormal electrical signals cause the heart to beat faster than normal, usually more than 100 beats per minute, with the beats starting in the heart ventricles. VT generally occurs in people with underlying heart abnormalities. VT can sometimes occur in structurally normal hearts, and in such patients the origin of abnormal electrical signals can be in multiple locations in the heart. One common location is in the right ventricular outflow tract (RVOT), which is the route the blood flows from the right ventricle to the lungs. In patients who have had a heart attack, scarring from the heart attack can create a milieu of intact heart muscle and a scar that predisposes patients to VT.

Other common causes for conduction disorders include defects in the left and/or right ventricle fast activation fibers, the His-Purkinje system, or scar tissue. As a result, the left and right ventricles may not be synchronized. This is referred to as Left Bundle Branch Block (LBBB) or Right Bundle Branch Block (RBBB).

Cardiac resynchronization therapy (CRT), also referred to as biventricular pacing or multisite ventricular pacing, is a known way to improve heart function in cases of LBBB or RBBB. CRT involves simultaneous pacing of the right ventricle (RV) and the left ventricle (LV) using a pacemaker. To implement CRT, a coronary sinus (CS) lead is placed for LV pacing in addition to a conventional RV endocardial lead (with or without a right atrial (RA) lead). The basic goal of CRT is to improve the mechanical functioning of the LV by restoring LV synchrony in patients with dilated cardiomyopathy and a widened QRS period, which is predominantly a result of LBBB.

Catheter ablation is a treatment of choice in patients with VT and/or symptomatic PVCs. The targets for ablation are locations in the heart where PVCs are occurring or locations where the onset of the VT is occurring. In order to determine a proper ablation location, a treating physician may first stimulate a proposed location using an electrical lead, in order to determine whether ablation at the proposed location will provide a desired electrical activation pattern stimulation of the heart.

Currently, determining the proper positioning of leads to obtain maximum cardiac synchronization or a desired electrical activation pattern involves a certain amount of guesswork on the part of an operating physician.

However, current methods do not allow for the determination of the optimal location for electrical leads, on a patient by patient basis. Further, if a desired activation pattern is not achieved when the heart is stimulated at a given location, current methods do not provide directional guidance for adjusting the lead location to provide an improved activation pattern. Accordingly, there is a need for improved guidance in determining the proper location for electrical leads for CRT and determining ablation locations.

### SUMMARY

Various embodiments provide improved cardiac imaging methods for determining locations for placing pacing electrodes within the heart.

Some embodiments provide cardiac imaging systems including fiducial markers, an external imaging device configured to generate image data of a patient's chest including the fiducial markers and electrocardiogram (ECG) leads placed on the patient's chest, and a processing unit configured to identify anatomical locations corresponding to the fiducial markers by detecting light reflected from the fiducial markers included in the image data.

Some embodiments provide fiducial marking kit including first fiducial markers configured to reflect a first wavelength of light, a second fiducial marker configured to reflect a second wavelength of light different from the first wavelength, a peelable backing. The first and second fiducial markers may be disposed on the peelable backing inside of the packaging.

Some embodiments provide cardiac imaging methods including applying fiducial markers and electrocardiogram (ECG) leads to a patient's chest, generating external image data of the patient's chest including the fiducial markers and the ECG leads, identifying identify anatomical locations corresponding to the fiducial markers by detecting light reflected from the fiducial markers included in the image data, and generating a patient-specific three dimensional (3D) anatomical model that merges the image data with a 3D anatomical model of the patient's chest by registering (i.e., aligning) the identified anatomical locations with corresponding anatomical locations in imaging obtained from CT or MRI scans.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is an example of a 3D model of a heart according to various embodiments.

FIG. 2A is a plan view of a 3D model of electrical activation of a heart according to various embodiments.

FIG. 2B is a plan view of a 3D model of electrical activation of a heart according to various embodiments.

FIG. 2C is a plan view of a synchronicity map according to various embodiments.

FIG. 2D is a plan view of a synchronicity map according to various embodiments.

FIG. 3 is a schematic representation of a cardiac imaging system according to various embodiments.

FIGS. 4A and 4B are 3D images of electrical leads and fiducial markers on a patient's torso according to various embodiments.

FIG. 4C is a schematic view of a fiducial marker kit according to various embodiments.

FIG. 5 is a flow diagram illustrating a cardiac imaging method using the system of FIG. 3, according to various embodiments.

FIG. 6A is a schematic representation of a cardiac imaging system according to various embodiments.

FIG. 6B is a flow chart illustrating a method according to various embodiments.

FIG. 7A is a top view non-limiting examples of fiducial marker according to an embodiment.

FIG. 7B is a side view of non-limiting examples of fiducial markers according to an embodiment.

FIG. 8 is an illustration of example fiducial markers applied to the torso according to an embodiment.

FIG. 9 is a top view of non-limiting examples of fiducial markers illustrating how shapes and colors may be varied so as to identify different anatomical features according to an embodiment.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

An electrocardiogram (ECG) is defined herein as any method that (preferably non-invasively) correlates actual electrical activity of the heart muscle to measured or derived (electrical activity) of the heart. In case of a classical electrocardiogram the differences in potential between electrodes on the body surface are correlated to the electrical activity of the heart. Derived ECG's can also be obtained in other ways (e.g. by measurement made by a so-called ICD (Implantable Cardioverter Defibrillator)). In order to obtain such a functional image an estimation of the movement of the electrical activity has to be provided.

Cardiac dyssynchrony has deleterious effects on cardiac function by depressing left ventricular (LV) mechanical performance, while increasing myocardial oxygen consumption. In addition, cardiac dyssynchrony probably causes LV remodeling. Therefore, cardiac dyssynchrony accelerates the progression of chronic congestive heart failure (CHF) and reduces patient survival.

During normal conduction, cardiac activation begins within both the left ventricular (LV) and right ventricular (RV) endocardium. In particular, electrical impulses (i.e., depolarization waves) travel substantially simultaneously through both the left and right ventricles. Bundle branch block (BBB) is a condition in which there is a delay or obstruction along the pathway of the electrical impulses. The delay or blockage may occur on the pathway that sends electrical impulses to the left or the right ventricles.

Left BBB is a condition in which the electrical impulses to the LV are slowed, and is one of the leading causes of cardiac desynchronization. In particular, activation begins only in the RV and proceeds through the septum before reaching the LV endocardium.

A pacemaker is an electronic device, approximately the size of a pocket watch, which senses intrinsic heart rhythms and provides electrical stimulation when indicated. Cardiac pacing can be either temporary or permanent.

Permanent pacing is most commonly accomplished through transvenous placement of leads to the endocardium (i.e., right atrium or ventricle) or epicardium (i.e., the LV surface via the coronary sinus), which are subsequently connected to a pacing generator placed subcutaneously in the infra-clavicular region. However, miniaturized pacemakers have been developed for implantation directly on or in the heart.

Cardiac resynchronization therapy (CRT) is a specialized type of pacemaker therapy that provides biventricular pacing. CRT is carried out with or without the use of an implantable cardioverter-defibrillator (ICD), a device employed for treatment and prophylaxis in patients at risk for ventricular tachycardia (VT) or ventricular fibrillation (VF).

In this application, areas in the heart that are electrically stimulated (e.g., paced) by a pacing electrode, micro catheter, or the like may be interchangeably referred to as a “pacing location” or a “stimulation location”.

Various embodiments include systems and methods for aiding physicians in placing pacing leads on a patient's heart for implementing CRT. Various embodiments include hardware systems including a processing unit configured with software to receive patient-specific data, generate and display a three-dimensional (3D) model of electrical activation of the heart in the form of a synchronicity map of the patient's heart based on electrocardiographic (ECG) imaging data, and correlate or register the 3D model/map with the patient's body using recognizable markers on the body that serve as fiducial reference points (referred to herein as “fiducial markers”). In particular, various embodiments may combine ECG signal information with a patient-specific 3D anatomical model of the heart, lungs, and/or torso identified with the markers in order to compute the positions of cardiac isochrones. The patient-specific 3D anatomical model may be obtained from an internal imaging system, such as a magnetic resonance imaging (MRI) device or computed tomography (CT), or with a 3D anatomical model with closes conformity to the patient. An external imaging system, such as a 3D camera, may be used to obtain 3D image data of the patient's body (e.g., the torso or chest) with key anatomical reference points (e.g., clavicles, shoulders, ribs, etc. indicated by the markers applied to the patient by a clinician as part of the set up for a CRT procedure. The patient-specific 3D anatomical model may merge the image data with a 3D anatomical model of the patient's chest by registering the identified anatomical locations with corresponding anatomical locations in imaging obtained from CT or MRI scans.

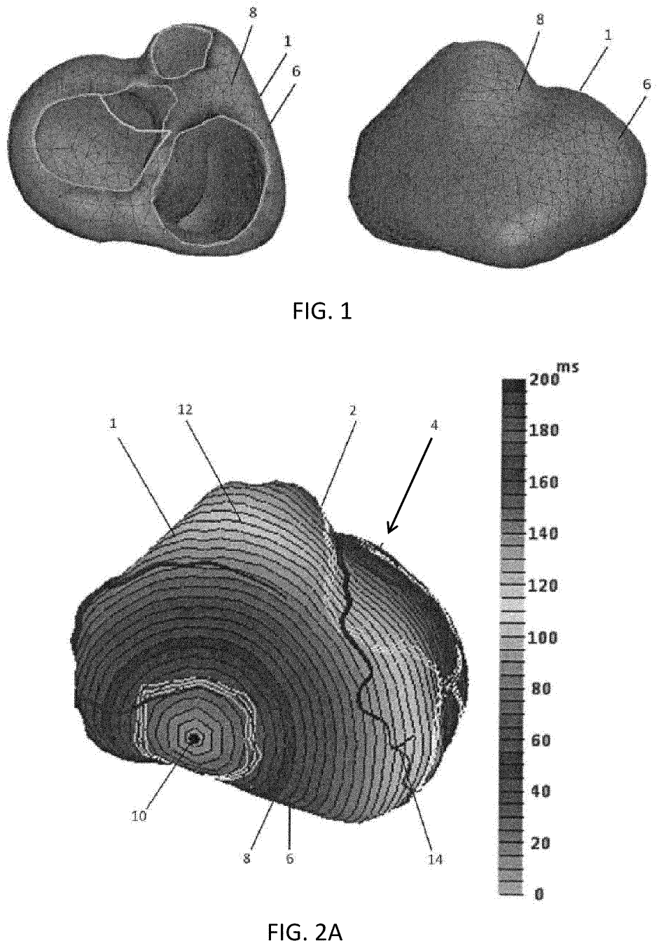

FIG. 1 shows a 3D model of a heart 1 seen in two different directions. The 3D model includes a mesh 6 representing an outer surface of the heart, here the myocardial surface. In this example, the model also may include the septal wall. The mesh 6 has a plurality of nodes 8. In this example, the mesh is a triangular mesh in which the surface of the heart is approximated by adjoining triangles.

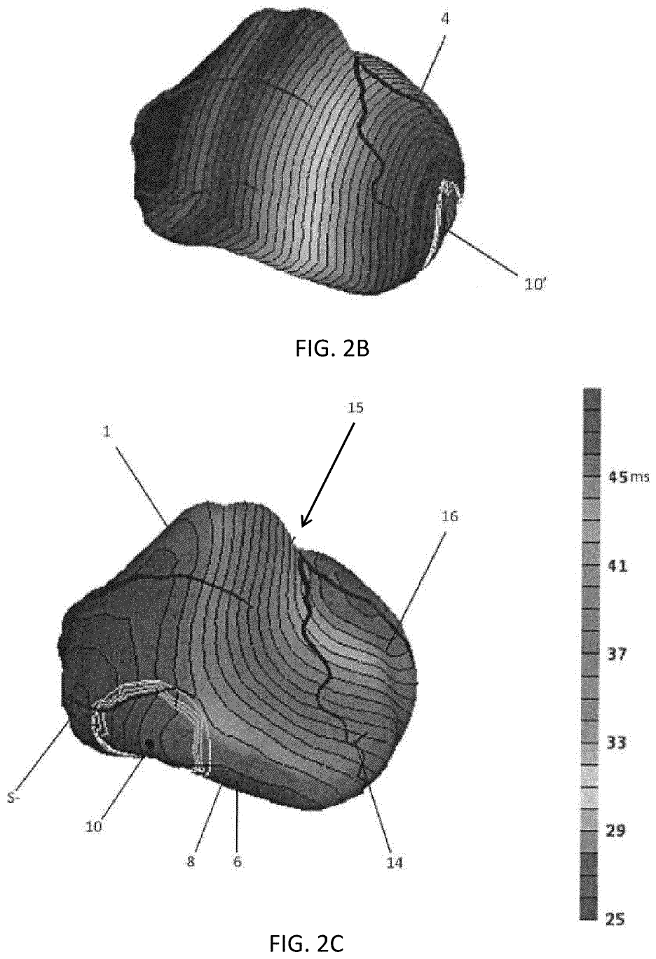

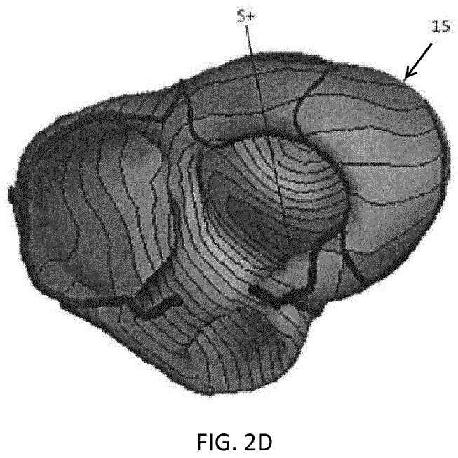

FIGS. 2A-2D are 3D models 4 of a heart showing the initial electrical activation of a heart 1 from various single stimulation location 10. FIGS. 2A-2C show a ventricular surface of the myocardium with a septal wall 2. In general, the 3D model 4 may include a mesh 6 representing a ventricular surface of the heart, here an outer surface of the ventricular myocardium with septal wall as represented in FIG. 1. The mesh 6 has a plurality of nodes 8. In the illustrated example, the heart 1 is electrically stimulated at a stimulation location 10. Upon electrical stimulation at the stimulation location 10, the electrical signals will travel through the heart tissue. Hence, different parts of the heart will be activated at different times. Each location on the heart has a particular delay relative to the initial stimulation. Each node 8 has associated therewith a value representative of a time delay between stimulation of the heart 1 at the stimulation location 10 and activation of the heart at that respective node 8. Locations that share the same delay time are connected by isochrones 12 in FIGS. 2A-2D. In this application, isochrones are defined as lines drawn on a 3D heart surface model connecting points on the model at which the activation occurs or arrives at the same time. The delay time for nodes across the heart surface in this example is also displayed by differing rendering shading. The vertical bar indicates the time delay in milliseconds associated with the respective colors. It will be appreciated that the stimulation location 10 can be the location of intrinsic activation of the heart 1.

The three dimensional model 4 of electrical activation of the heart 1 can also include further information. In the example of FIG. 2A, the model 4 includes information on cardiac blood vessels, in particular cardiac veins. From the patient-specific three-dimensional anatomical model of the heart information can be obtained on the position of blood vessels on the myocardium. This information is added to the 3D model 4 of electrical activation in that nodes are indicated as being associated with such blood vessel. The blood vessel 14 is then identified and optionally visible in the model 4 of electrical activation of the heart 1.

FIG. 2B shows an example 3D model resulting from initial stimulation at another stimulation location 10′. It will be appreciated that a view resulting from initial stimulation at other nodes of the mesh 6 may be generated for each node of the mesh 6.

A particular electrical activation sequence of the entire heart 1, resulting from stimulation at a particular node, may be summarized in a single parameter, namely, heart activation synchronicity. The heart activation synchronicity provides an indication of how synchronously the entire heart is activated. For common situations, a more synchronous activation of the heart is considered beneficial. The measure for heart activation synchronicity in this example is the standard deviation (std) of the depolarization (dep) times of the heart. Hence, the heart activation synchronicity provides an indication of synchronicity of activation of the entire heart as a result of stimulation at the respective node.

FIG. 2C shows an example of a heart synchronicity map 15. In the example illustrated in FIG. 2C, heart activation synchronicity is indicated for each node in the map 15. In this example, the indication may be shown by providing false colors and/or iso-sync lines 16. The iso-sync lines 16 connect nodes having the same heart activation synchronicity. The heart synchronicity map 15 provides a singular 3D overview showing the locations on the heart that result in good heart activation synchronicity, and the locations on the heart that result in poor heart activation synchronicity, if the heart were stimulated at such locations.

In the example illustrated in FIG. 2C, it can be seen that the original stimulation location 10 does not provide particularly good synchronization, with a heart activation synchronicity value of approximately 45 ms standard deviation of the depolarization times of the heart. The least favorable stimulation location, here the location with the highest heart activation synchronicity value, is indicated at S−. In this example, the most favorable stimulation location, where the lowest heart activation synchronicity value occurs, is indicated at S+. In some instances, the most favorable stimulation location S+ can best be seen when looking at the synchronicity map 15 from another direction, as shown in FIG. 2D.

The 3D model may also include further information. In the example illustrated in FIG. 2A, a 3D model 4 may include cardiac blood vessels 14 and/or veins on the myocardium. This information may be added to the 3D model 4 in that nodes are indicated as being associated with such blood vessel. The blood vessels 14 may then be identified and optionally shown in the 3D model 4. The 3D model 4 may also include information on scar tissue. Scar tissue locations may be obtained from delayed enhancement MRI images and added to the 3D model 4. Scar tissue can be simulated in the 3D model 4 by reducing the propagation velocity of electrical signals there through. Scar tissue can also be accounted for by selling the transition from one node to another to very slow or non-transitional for the areas in the heart wall where scar tissue is present.

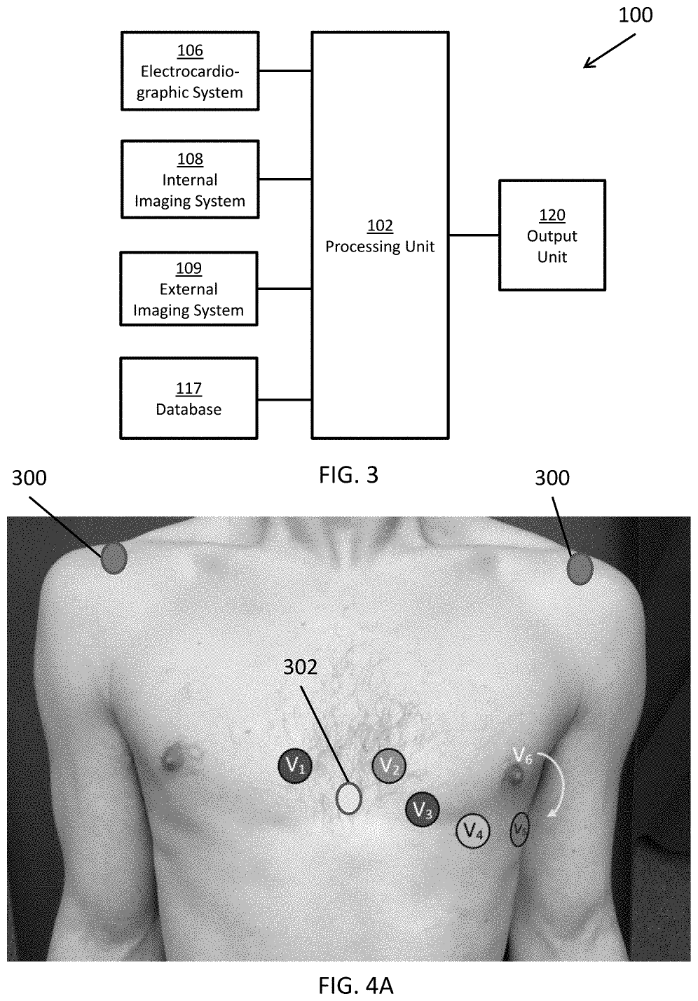

FIG. 3 is a system block diagram of a cardiac imaging system 100, according to various embodiments of the present disclosure. Referring to FIG. 3, the system 100 includes a processing unit 102 which may be electrically connected to hardware modules, such as an electrocardiographic system 106, an internal imaging system 108, an external imaging system 109, and an output unit 120.

The processing unit 102 receives patient-specific data from the hardware modules. From the patient-specific anatomical data, the processing unit 102 may generate a synchronicity map of the patient's heart, which may be output to the output unit 120. The output unit 120 may be configured to output the synchronicity map and/or alternative data to a user. The output unit may be a display unit, a printer, a messaging unit, or the like.

For example, the processing unit 102 may receive ECG imaging data from the electrocardiographic system 106, such as a 12 lead ECG device. The ECG data may be used by the processing unit 102 for determining the 3D model 4 of electrical activation of the heart. In particular, ECG signals may be combined with a patient-specific 3D anatomical model of the heart, lungs, and/or torso, in order to compute the positions of the cardiac isochrones.

The patient-specific 3D anatomical model may be obtained from the internal imaging system 108, such as an MRI device or CT device. Alternatively or additionally, a 3D anatomical model showing closest conformity to the patient may be selected, and optionally modified, from a database including a plurality of 3D anatomical models. The selected, and optionally modified, 3D anatomical model may serve as the patient-specific 3D anatomical model.

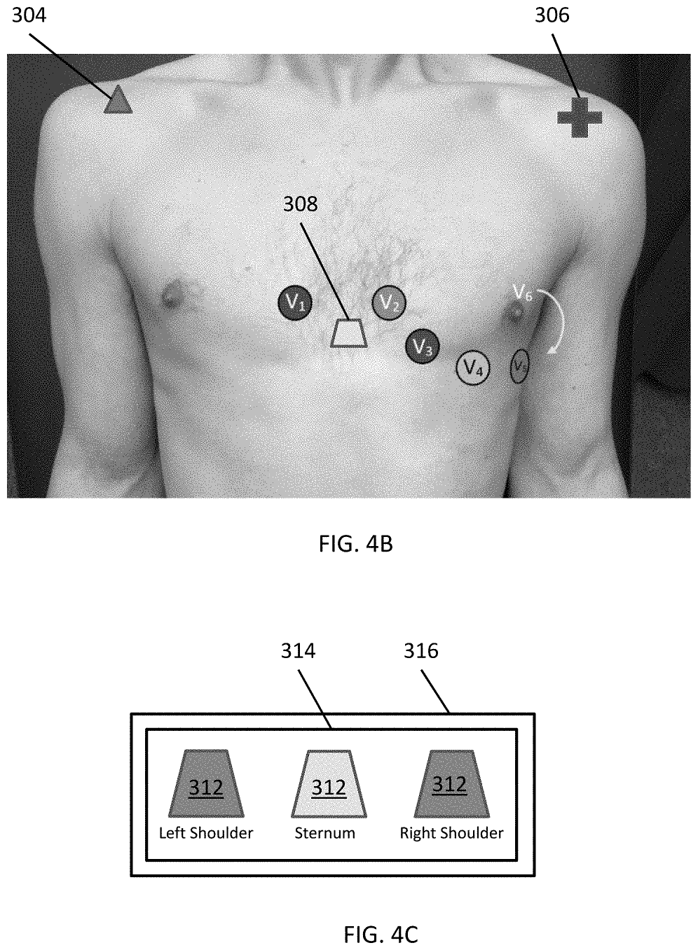

Further, the processing unit 102 may receive patient image data from the external imaging system 109. For example, the external imaging system 109 may be 3D camera, and the processing unit 102 may receive 3D image data of the surface a patient's chest, as shown in FIG. 4A or 4B.

Referring to FIG. 4A, the 3D image data may include the positions of ECG leads relative to the anatomy of the patient, such as the V1-6 precordial electrodes shown in FIG. 4A. Knowledge of the location of the ECG electrodes relative to the heart, and in particular the V1-6 precordial electrodes, may be especially important for accurately computing the onset location of PVC.

In some embodiments, the offsets of the electrodes from their assumed ideal locations, and in particular offsets of the V1-6 electrodes, may be determined based on a comparison of detected ECG signals of a normal heart beat to ideal ECG normal heart beat signals. For example, the offsets may be determined based on how a detected ECG signal will be affected by variations in the position of electrodes with respect to ideal electrode positions. In particular, the recorded ECG data may be used to determine a stimulation onset location for a normal beat. Since the normal onset location in the SA node is known, the determined offset location may be compared to this known onset location, and the offset of the electrodes may be deduced based on the variation therebetween. As such, it may be possible to determine electrode offsets without generating the 3D map.

The processing unit 102 may be configured to align and/or merge the 3D image data generated by the external imaging system 109 and the anatomical torso and/or heart model generated by the internal imaging system 108, and the locations of the electrodes in the torso model may be adjusted to coincide with the electrode locations in the 3D image data. However, if the external imaging system 109 is not properly aligned with the torso, it may be difficult to properly the 3D image data and the anatomical model.

In order to facilitate the alignment of the 3D image data and the anatomical torso model, the system 100 may include fiducial markers placed on (e.g., adhered to) a patient's torso prior and captured in the 3D image data generated by the external imaging system 109. The fiducial markers may be placed on the patient by a clinician in set anatomical locations that are identified in the torso model in order to facilitate alignment of the 3D image of the patient with the anatomical torso model. In some embodiments, the fiducial markers may be stickers having an adhesive backing configured to adhere to the skin, with a shape, color and/or surface material (e.g., reflective or retroflective material) that enables automatic identification and location of the markers by a processor processing the 3D image data.

For example, first fiducial markers 300 may be placed on the patient's shoulders at set anatomical locations, such as at the distal end of each clavicle. A second fiducial marker 302 may be placed at a set anatomical location between the first fiducial markers 302, such as at a set position on the patient's sternum.

The processing unit 102 may be configured to identify the fiducial markers 300, 302, and anatomical locations corresponding thereto, based on one or more identifying characteristics thereof included in the 3D image data collected by an external imaging device. In some embodiments, the processing unit 102 may be configured to identify anatomical locations corresponding to the fiducial markers 300, 302, based on the color, shape, and/or reflectivity of the corresponding anatomical markers included in the image data.

In some embodiments, the fiducial markers 300, 302 may be configured to reflect specific wavelengths of light. In some embodiments, the first fiducial markers 300 may have a first color and the second fiducial marker 302 may have a second color. In some embodiments, each marker 300, 302 may have a different color.

In some embodiments the fiducial markers 300, 302 may include a reflective material, which may be in the form of a reflective coating. In some embodiments, the reflective material may be configured to reflect one or more specific wavelengths, or wavelength ranges, of light. For example, in some embodiments the fiducial markers 300, 302 may be formed of materials configured to reflect visible light, infrared light, ultraviolet light, or a combination thereof. In some embodiments, the external imaging system 109 may include a light source, and the reflective material may be configured to reflect all or some of the light emitted from the light source. For example, the fiducial markers 300, 302 may be configured to selectively reflect particular wavelengths or wavelength ranges of the emitted light. The processing unit 102 may be configured to identify the fiducial markers 300, 302 based on the light reflected thereby.

In some embodiments, the fiducial markers 300, 302 may include a retroflective material. In particular, the retroflective material may be configured to reflect incident light, or a portion thereof, at an angle substantially equal to the angle of incidence of the incident light (i.e., directly back towards the source of the incident light). Retroflective materials are well known as used in safety vests and on traffic signs, for example. In such embodiments, the processing unit 102 may be configured to detect such light as a luminosity peak in the image data received from the external imaging system.

In some embodiments, the fiducial markers may have one or more different shapes. For example, as shown in FIG. 4B, the system 100 may include triangular fiducial marker 304, a cross-shaped fiducial marker 306, and/or a trapezoidal fiducial marker 308. The processing unit 102 may be configured to identify anatomical locations corresponding to the fiducial markers, based on the shapes thereof.

However, various embodiments are not limited to any particular fiducial marker identifying characteristics, so long as the fiducial markers include a characteristic identifiable by the processing unit 102 and detectable by the external imaging system 109. Further, while three fiducial markers are shown in FIGS. 4A and 4B, any suitable number of fiducial markers may be used.

In some embodiments, fiducial markers may be included in a kit. FIG. 4C illustrates a kit 310 including fiducial markers 312. Referring to FIG. 4C, the kit 310 may include fiducial markers 312 sufficient for marking one patient. For example, kit 310 may include a peelable backing 314 on which the fiducial markers 312 are disposed, and packaging 316. In some embodiments, the packaging 316 may be sterile. The backing 314 may include indicia identifying the fiducial markers by intended location.

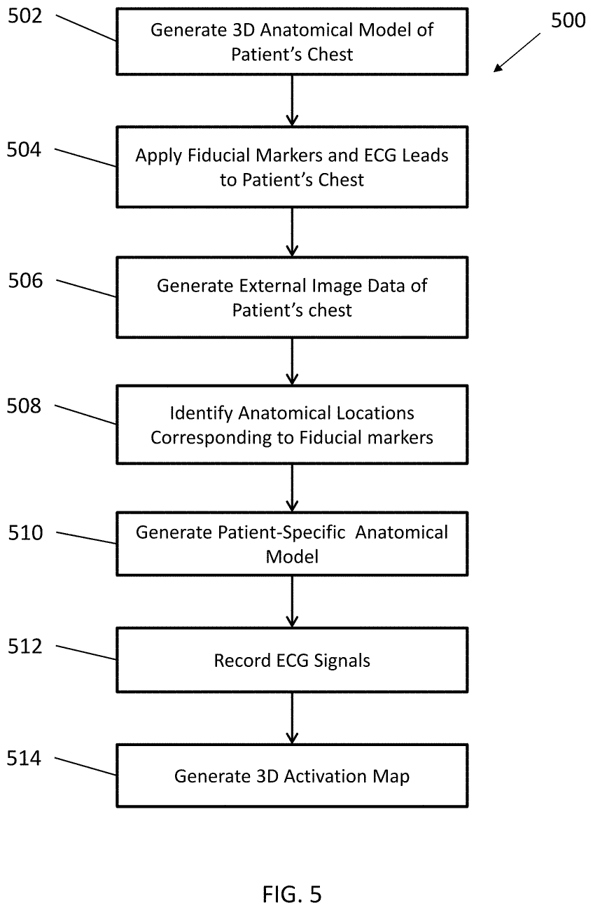

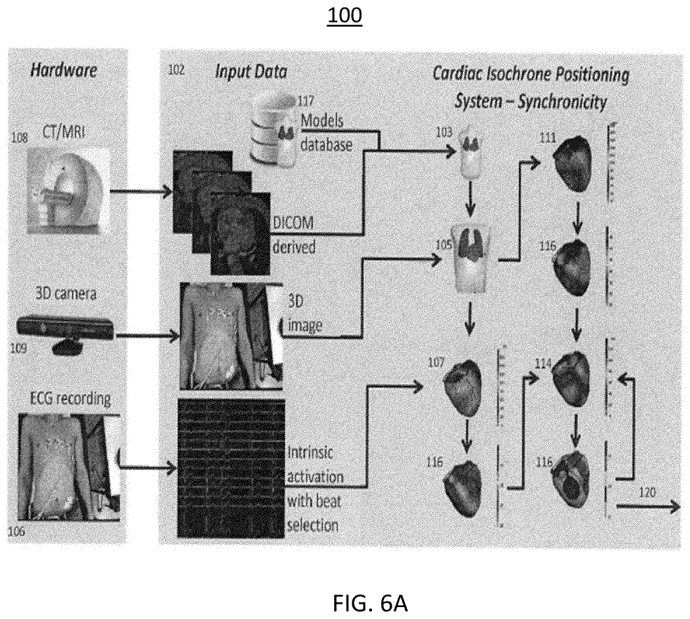

FIG. 5 is a flow diagram illustrating a cardiac imaging method using the system 100, according to various embodiments of the present disclosure. Referring to FIG. 5, in block 502, a 3D anatomical model of a patient's chest is generated. In particular, the model may be generated using a CT device, an MRI device, or may be selected from a database.

In block 504, ECG leads and fiducial markers are applied to the patient's chest. In particular, the fiducial markers may be applied to the shoulders and sternum of the patient by a clinician as part of preparing for a procedure as described above.

In block 506, an external image of the chest by an external imaging device, such as a 3D camera, to generate 3D image data. In particular, the external image data is configured to record the positions of the ECG leads and the fiducial markers on the patient.

In block 508, the processing unit 102 identifies anatomical locations corresponding to the fiducial markers by identifying and locating the fiducial markers within the 3D image data based on the markers' corresponding characteristics as described above. For example, the processing unit 102 may identify anatomical locations based on shapes and/or particular characteristics of light reflected from corresponding fiducial markers.

In block 510, the processing unit 102 generates a patient-specific anatomical model of the patient by merging the anatomical image or model with the external 3D image data. In particular, the processing unit 102 may use the locations of the identified fiducial markers on the patient to align the corresponding anatomical features with corresponding locations in the anatomical image obtained from CT or MRI scans or a model so that the locations of the ECG leads may be accurately determined.

In block 512, ECG signals from the patient are recorded. In block 514, the processing unit 102 uses the ECG signals to generate a 3D activation map of the patient's heart. In particular, the processing unit 102 may generate the activation map based on the ECG signals and the locations of the ECG leads and the 3D heart model provided in the patient-specific anatomical model.

According to various embodiments of the present disclosure, the method steps may be performed in any suitable order. For example, the ECG signals may be recorded before the 3D anatomical model is generated.

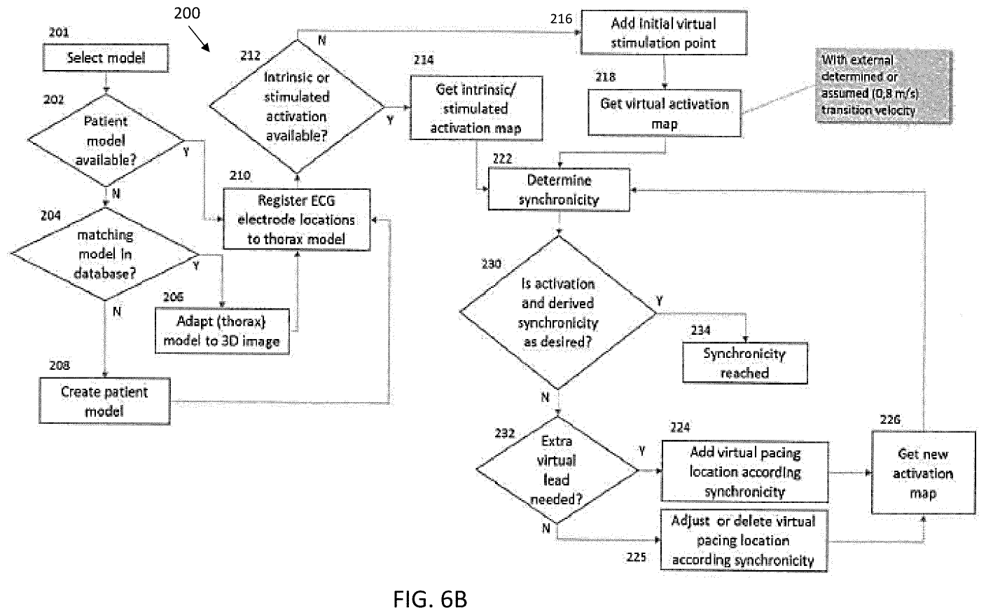

FIG. 6A is a data flow representation of further operations of the system 100, and FIG. 6B is a flow diagram illustrating a method 200 of operating the system 100, according to various embodiments of the present disclosure. Referring to FIGS. 6A and 6B, processing unit 102 may include various sub-systems, and may perform the operations of the method 200 to generate a synchronicity map.

In the method 200, the processing unit 102 may use a patient-specific 3D anatomical model of the thorax of the patient and the size, orientation, and location of the size, orientation, and location of the heart within the thorax. Such a model may be selected in block 201 for further use by the processing unit 102. The processing unit 102 may determine whether such a model is already available in determination block 202. If the model is not yet available (i.e., determination block 202=N), a retrieval unit 103 may check whether a suitable anatomical model for this patient is present in a database 117 in determination block 204.

If no suitable patient-specific anatomical model is available in the database 117 (i.e., determination block 202=N), the retrieval unit 103 may generate the patient-specific anatomical model on the basis of the received patient-specific anatomical 3D image data in block 208.

If a suitable patient-specific anatomical model is available in the database 117 (i.e., determination block 202=Y), the retrieval unit 103 retrieves the suitable anatomical model from the database 117 in block 206. Also in block 206, the retrieval unit 103 may adapt the anatomical model from the database to the 3D image of the patient, so as to transform the selected anatomical model into a (quasi) patient-specific 3D anatomical model. Optionally, the patient-specific 3D model may also include the size, orientation and/or location of other structures in the patient, such as the lungs and/or other organs. The patient-specific 3D model may be a volume conductor model. The retrieval unit 103 may also detect the locations of fiducial markers in the 3D image and use the detected locations to adapt the 3D image to the anatomical model.

If a patient model is available (i.e., determination block 202=Y), or using a patient model created in block 208 or a stored model adapted to the patent in block 206, the positions of ECG leads and the patient-specific model, a lead locator module 105 may determine corresponding positions of the ECG leads in the patient-specific 3D model to provide an enhanced patient-specific model in block 210.

In determination block 212, when the patient-specific anatomical model and/or the enhanced patient-specific model available, a determination is made as to whether ECG data representative of intrinsic or stimulated activation is available. If intrinsic activation data or pacing stimulation from one or more already present pacemaker leads is available (i.e., determination block 212=Y), an activation unit 107 may generate a 3D electrical model of showing the current activation of the heart of the patient on the basis of the patient-specific model and the ECG data in block 214.

If no ECG data on intrinsic or stimulated activation is available (i.e., determination block 212=N), a virtual stimulation unit 111 may add an initial virtual stimulation to an electrical model of the heart, based on previously determined and/or assumed transition velocities between nodes, in block 216. An assumed transition velocity may be 0.8 ms, for example. The electrical model may include arteries, veins, and/or scar tissue as explained above. In block 218, a 3D electric model of virtual activation of the heart of the patient may be generated.

From the 3D electric model of intrinsic, stimulated, or virtual activation of the heart of the patient, a synchronicity determination unit 116 may generate a synchronicity map 15 in block 222, as described above. On the basis of the synchronicity map, the processing unit 102 may determine whether the artificial stimulation location or virtual stimulation location resulted in optimal activation and synchronicity in determination block 230. If so (i.e., determination block 230=Y), the processing unit may calculate optimal stimulation locations for a patient's heart in block 234.

If it is determined in block 230 that optimum synchronicity has not been reached (i.e., determination block 230=N), the method 200 proceeds to determination block 232 in which it is determined whether an extra virtual stimulation location is needed or should be added, or if a virtual stimulation location should be moved or changed with respect to the timing parameters. This determination may be made by a clinician, by the processing unit, or by the clinician based on information or recommendations presented on a display by the processing unit.

If it is determined that an extra virtual lead is needed (i.e., determination block 232=Y), a virtual pacing location may be added according to the determined synchronicity in block 224. If it is determined that an extra virtual lead is not needed and a virtual stimulation location should be moved or changed (i.e., determination block 232=N), the artificial or virtual stimulation location may be adjusted accordingly in block 225.

In block 226, a new activation may be generated. Synchronicity may then be recalculated in block 222, and the process may be repeated until a desired activation is determined to be achieved in determination block 230.

The system 100 may also virtually adapt the current artificial stimulation locations, i.e., pacemaker lead locations, with respect to its current stimulation parameters to reach optimum synchronicity.

The system 100 may also be used for assessing multiple stimulations. For example, the multiple stimulations may be a combination of intrinsic activation and stimulated activation (pacing). For example, the multiple stimulations may be a multiple stimulated activation (pacing). It is possible that the user or the processing unit 102 determines 232 whether an additional stimulation location, such as an additional pacemaker lead, would be desirable.

If an additional stimulation location is desired, an additional stimulation location may be inserted by the insertion unit 114. Then, activation for the situation with the original stimulation location and the added virtual stimulation location may be determined again in block 226, and synchronicity may be recalculated in block 222. On the basis of the synchronicity map, the processing unit 102 may determine in determination block 230 whether the additional virtual stimulation location resulted in optimum synchronicity. If the optimum synchronicity has not been reached, the method 200 proceeds to block 232, in which it is determined whether an extra virtual stimulation location should be added, or if a virtual stimulation location should be moved or removed, with respect to the timing parameters. In such a case, the process may be repeated one or more times.

Based on the patient specific cardiac activation model, a cardiac synchronicity model may be generated. The synchronicity model may be a 3D heart surface model including iso-sync lines as described above in which the iso-sync lines represent the activation synchronicity of the heart. This synchronicity may be based on specific activation conditions, such as right ventricle activation at a lead position of a pacemaker.

As an example, the synchronicity model may be generated and the activation isochrones for the intrinsic LBBB pattern may be determined in the following blocks.

1A) A patient-specific anatomical 3D model of the heart, lungs, and thorax may be generated, such as on the basis of an MRI or CT image of the patient, or derived from a model taken from a database adapted to the patient's dimensions, such as with use of the 3D camera. The anatomical 3D model imaging obtained from CT or MRI scans or otherwise may include a 3D surface model of the heart, a 3D surface model of the lungs, and a 3D surface model of the thorax. A 3D surface model may be a close approximation of the actual surface of the heart by means of a mesh of a plurality of polygons, such as triangles, connected at their corners. The interconnected corners form nodes of the mesh.

1B) An ECG, e.g. a 12-lead ECG, may be measured. The locations of the electrodes of the ECG device on the thorax may be recorded. The positions of the electrodes in the 3D anatomical model are used for estimating the distribution, fluctuation, and/or movement of electrical activity through heart tissue. The locations of the recording leads or the ECG device may be entered in the anatomical 3D representation of the thorax.

1C) Optionally, scar tissue may be incorporated in the anatomical 3D representation of the heart. The presence and location of scar tissue may be derived from delayed enhancement MRI images.

1D) The measurements per recording lead of the ECG device may be related to the heart and torso geometry. Using an inverse procedure, the intrinsic activation may be determined. The distribution, fluctuation, and/or movement of electrical activity through heart tissue may be based upon a myocardial distance function, a fastest route algorithm, shortest path algorithm, and/or fast marching algorithm.

2) Once the activation isochrones for the intrinsic LBBB pattern have been determined, a stimulus site may be added to the intrinsic activation for each node on the heart and the desired synchronicity of the heart may be computed from the outcome. A “node” refers to an intersection point of the triangles of upon which the anatomical 3D heart model is based.

The above methods may also be used to determine an optimal location for placement of a cardiac pacemaker electrode. To determine the optimal pacing site, synchronicity maps may be computed. The intrinsic activation map, in combination with a determined stimulation point may be applied to a new cardiac isochrone positing map.

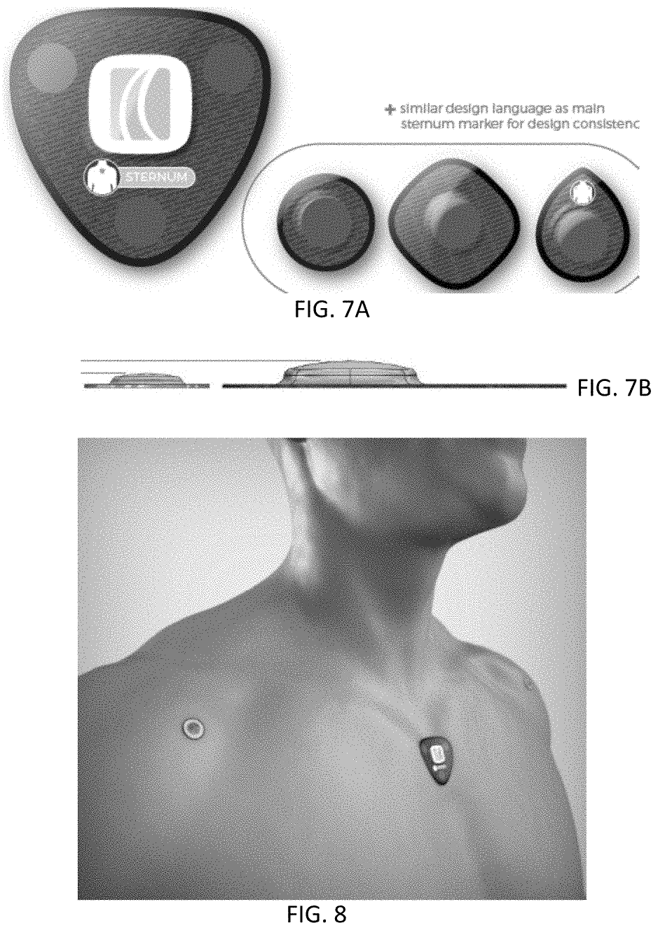

FIG. 7A illustrates some examples of fiducial marker according to an embodiment. As illustrated, fiducial markers may have different shapes, sizes and colors to enable an imaging and processing system to recognize the markers and the anatomical feature to which they are applied. For example, markers may have triangular, square, round or other shapes, such as tear drop. Further markers may use color patterns to enable an imaging/processing system to recognize and locate the markers within image data. For example, the illustrated marks include a red dot on a blue background. This color combination may be recognizable and the red dot may be used by a system to locate the underlying anatomical feature.

FIG. 7B is a side view of non-limiting examples of fiducial markers to illustrate that the markers may be flat or include some thickness to provide a 3D image.

FIG. 8 illustrates an example of how fiducial markers may be applied to the torso of a patient. In the illustrated example, a large marker may be placed on the sternum, and include multiple dots, which may define the dimensions and orientation of the sternum. Smaller markers may be applied to other anatomical features, such as the front of the shoulder as illustrated.

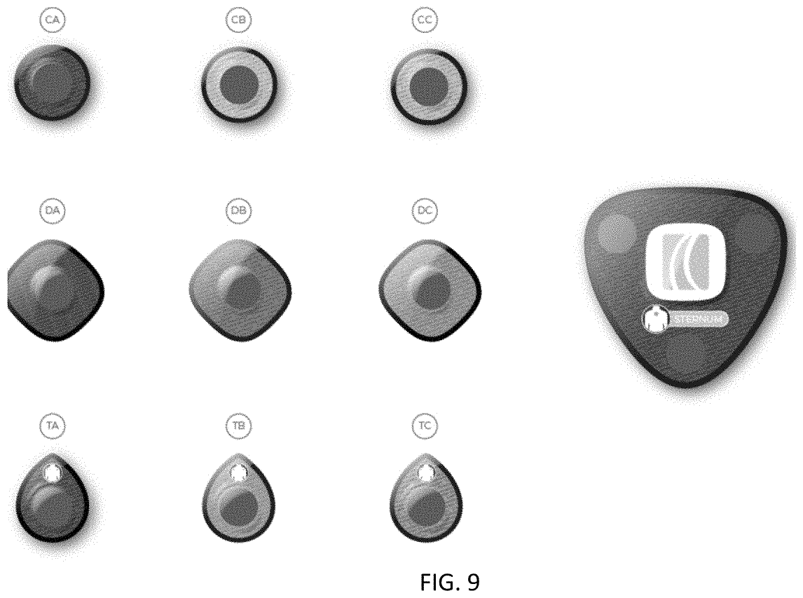

FIG. 9 illustrates a non-limiting example of how fiducial markers may be provided (e.g., in a kit) with different shapes and colors to enable a clinician to mark several different parts of a patient's body with the shape and color patterns enabling an imaging/processing system to identify or recognize the different anatomical features. For example, a clinician applying fiducial markers to a patient may enter information into the system to associate particular structures (e.g., shoulder, clavicle, ribs, etc.) to particular shapes and background colors.

In some embodiments, electrodes may similarly be identified with shapes and colors different from the fiducial markers so that an imaging/processing system can also distinguish markers from electrodes and record the locations of electrodes with respect to the markers.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof. If implemented in software, the functions may be stored as one or more instructions or code on a non-transitory computer-readable medium or non-transitory processor-readable medium. The steps of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What is claimed is:

1. A cardiac imaging system comprising:
fiducial markers configured to be placed on a patient and configured to be recognizable using image processing;
an external imaging device configured to generate image data of the patient's body including the fiducial markers and electrocardiogram (ECG) leads placed on the patient's chest; and
a processing unit configured to identify anatomical locations corresponding to the fiducial markers by detecting light reflected from the fiducial markers included in the image data.

2. The system of claim 1, wherein:
the fiducial markers are configured to reflect a first wavelength of light or a second wavelength of light different from the first wavelength; and
the processing unit is configured to identify the anatomical locations based on whether the corresponding fiducial marker reflects the first wavelength or the second wavelength.

3. The system of claim 2, wherein the fiducial markers comprise:
first and second fiducial markers configured to reflect the first wavelength; and
a third fiducial marker configured to reflect the second wavelength.

4. The system of claim 1, wherein:
the fiducial markers are individually configured to preferentially reflect a first wavelength of light, a second wavelength of light, or a third wavelength of light; and
the processing unit is configured to identify the anatomical locations based on whether the corresponding fiducial marker reflects the first wavelength, the second wavelength, or the third wavelength.

5. The system of claim 4, wherein the fiducial markers comprise:
a first fiducial marker configured to reflect the first wavelength;
a second fiducial marker configured to reflect the second wavelength; and
a third fiducial marker configured to reflect the third wavelength.

6. The system of claim 1, wherein:
the fiducial markers have a first shape or a second shape;
the processing unit is configured to identify the anatomical locations based on whether the corresponding fiducial marker has the first shape or the second shape.

7. The system of claim 1, wherein:
the fiducial markers have a first shape, a second shape, or a third shape;
the processing unit is configured to identify the anatomical locations based on whether the corresponding fiducial marker has the first, second, or third shape.

8. The system of claim 1, wherein the anatomical locations are located on the shoulders and the sternum of the patient.

9. The system of claim 1, wherein the fiducial markers are configured to adhere to the skin of the patient.

10. The system of claim 1, wherein the processing unit is configured generate a patient-specific three dimensional (3D) anatomical model merging the image data with a 3D anatomical model of the patient's chest by registering the identified anatomical locations with corresponding anatomical locations in imaging obtained from CT or MRI scans.

11. The system of claim 10, wherein the 3D anatomical model is generated based on magnetic resonance (MRI) images or computed tomography (CT) images of the patient's chest.

12. The system of claim 1, wherein the external imaging device is a three dimensional (3D) camera.

13. A fiducial marking kit comprising:
first fiducial markers configured to reflect a first wavelength of light;
a second fiducial marker configured to reflect a second wavelength of light different from the first wavelength;
a peelable backing; and
packaging,
wherein the first and second fiducial markers are disposed on the peelable backing inside of the packaging.

14. A cardiac imaging method comprising:
applying fiducial markers and electrocardiogram (ECG) leads to particular anatomical locations on a patient's body;
generating external image data of the patient's body including imaging the fiducial markers and the ECG leads;
identifying anatomical locations corresponding to the fiducial markers by detecting light reflected from the fiducial markers included in the image data; and
generating a patient-specific three dimensional (3D) anatomical model merging the image data with a 3D anatomical model of the patient's chest by registering the identified anatomical locations with corresponding anatomical locations in imaging obtained from CT or MRI scans.

15. The method of claim 14, wherein identifying anatomical locations corresponding to the fiducial markers comprises detecting two different wavelengths of light reflected from two different fiducial markers.

16. The method of claim 15, wherein:
two of the fiducial markers are placed on the shoulders of the patient; and
one of the fiducial markers is placed on the sternum of the patient.

17. The method of claim 14, wherein identifying anatomical locations corresponding to the fiducial markers comprises detecting three different wavelengths of light reflected from three different fiducial markers.

18. The method of claim 14, wherein:
the fiducial markers have different shapes; and
the identifying anatomical locations corresponding to the fiducial markers by detecting light reflected from the fiducial markers included in the image data comprises detecting the shape of each fiducial marker.
